(19) United States
(12) Patent Application Publication
(10) Pub. No.: US 2012/0133752 A1
(43) Pub. Date: May 31, 2012

(54) APPARATUS FOR POSITIONING A PERCUTANEOUS ACCESS DEVICE

(76) Inventor: John Bry, Richmond, CA (US)

(21) Appl. No.: 13/304,138

(22) Filed: Nov. 23, 2011

Related U.S. Application Data
(60) Provisional application No. 61/417,005, filed on Nov. 24, 2010.

(51) Int. Cl. H04N 7/18; A61B 1/00

(52) U.S. Cl. 348/65; 600/114; 348/E07.085

(57) ABSTRACT

Embodiments disclosed herein relate to medical procedures. Particularly disclosed are systems and methods for positioning a percutaneous access device for use with imaging sensors. A positioning apparatus is described, which may comprise at least one imaging device coupled to a guide device. The guide device may comprise a base plate and a guide member pivotally coupled to the base plate. The guide member may be configured with an alignment feature in which a percutaneous access device may rest. The at least one imaging device may comprise an imaging probe or a docking station configured to receive an imaging probe. Movement of the guide member and the one or more imaging devices may be coupled to facilitate imaging of sub-surface structures toward which the guide member and accompanying percutaneous access device are angled. A method of using a positioning apparatus to position a percutaneous access device is also described.

### CROSS-REFERENCE TO RELATED APPLICATIONS

This application claims the benefit of U.S. Provisional Application No. 61/417,005, which was filed Nov. 24, 2010, and is entitled “ULTRASONIC PERCUTANEOUS ACCESS DEVICE.” The entire disclosure of this provisional application is incorporated herein by reference in its entirety.

### BACKGROUND OF THE INVENTION

1. Field of the Invention

Embodiments disclosed herein relate to medical procedures. More specifically, certain embodiments relate to an apparatus for positioning a percutaneous access device and methods of using such an apparatus.

2. Description of the Related Art

Many medical procedures require access to a patient's vasculature. Such procedures include, but are not limited to, intravenous administration of fluids and nutrients, administration of chemotherapy drugs, antibiotics, and other intravenous medications, hemodialysis, blood withdrawal for diagnostic testing, and transfusions of blood or blood products. Vascular access is most commonly obtained by puncturing a patient's skin and advancing a needle through subcutaneous tissue and the wall of a target blood vessel. The tip of the needle must be positioned so as to penetrate into the lumen of the blood vessel without advancing into the other side of the vessel wall. The depth and dimensions of blood vessels are not directly visible at the skin's surface; therefore, it can be quite challenging to determine the exact location of a target blood vessel and gain the necessary vascular access. Obesity, narrowing of blood vessels, atypical anatomies, and vessel scarring are all conditions that may increase the difficulty of gaining vascular access.

To aid in the proper placement of vascular access devices, medical professionals often utilize X-ray imaging techniques. Placement of the vascular access device may occur in a surgical suite under the guidance of an X-ray machine, or it may be performed free-hand at the patient's bedside with X-ray images taken subsequently to confirm the proper placement of the vascular access device. Use of X-ray imaging is not ideal as it exposes the patient to radiation. Moreover, moving the patient to special X-ray equipped rooms may increase wait time, procedure time, and costs.

Ultrasound technology can be used to image some percutaneous structures. Imaging with ultrasound is often utilized to facilitate proper placement of biopsy needles. One of the most widely used methods of biopsy procedures is the “free-hand” technique. In performing such a technique, a medical professional positions an imaging probe containing an ultrasonic sensor with one hand while manipulating the placement of the biopsy needle with the other hand. The ultrasonic sensor provides an image of the percutaneous structures within the imaging plane of the sensor. In order to facilitate needle placement, the needle must be carefully positioned within the sensor's narrow imaging plane. Considerable skill is required, and the procedure often results in repeated needle punctures before proper needle placement is achieved. Accordingly, a need exists for a positioning guide that may be used to facilitate vascular access procedures.

### SUMMARY

The systems, methods and devices of the disclosure each have several innovative aspects, no single one of which is solely responsible for the desirable attributes disclosed herein.

In one embodiment, an apparatus is configured to guide the positioning of a percutaneous access device for highly accurate percutaneous vascular access, needle aspiration, core biopsy or nerve blockade. The apparatus includes a guide device comprising a base plate and a guide member. The guide member has a first surface and a second surface opposite the first surface and is pivotally coupled to the base plate such that the second surface is proximal to the base plate. The guide member can also have an alignment feature disposed on the first surface which is configured to receive and guide a percutaneous access device along a first direction on the guide member towards a target point. In some embodiments, the alignment feature is disposed in a direction parallel to a longitudinal axis of the guide member, and in one embodiment, the alignment feature is a groove. In other embodiments, the alignment feature is one of at least one guide hook, a notch, a tube, a trough between two ridges, and a groove.

In several embodiments, the apparatus also includes at least one imaging device that is coupled to the guide device and positioned to image an area around the target point. The at least one imaging device can be rotationally coupled to the guide device. In some embodiments, the at least one imaging device includes an ultrasonic probe positioned at a leading edge of the guide device. In other embodiments, the at least one imaging device includes a housing for holding an imaging probe. In one embodiment, the at least one imaging device includes two imaging devices disposed apart from each other, wherein the two imaging devices are both configured to image an area around the target point, each configured to image from a different position. The two imaging devices can be coupled to the guide device and configured to move as the guide member is pivotally moved relative to the base plate. In some embodiments, the coupling of the two imaging devices to the guide device includes a plurality of toothed cogs on the imaging devices, which are configured to engage a plurality of toothed cogs on the guide device. Movement of the imaging devices changes the imaging area of each device.

In several embodiments, the apparatus also includes an attachment device coupled to the base plate to removably secure the guide device to an extremity. In one embodiment, the attachment device includes a strap.

In another embodiment, a method of guiding the insertion of a percutaneous access device includes positioning a guide device on an extremity. The guide device can include a base plate coupled to a guide member. The guide member of the guide device has a first surface and a second surface opposite the first surface and is pivotally coupled to the base plate such that the second surface is proximal to the base plate. The guide member can also have an alignment feature disposed on the first surface that is configured to receive and guide a percutaneous access device towards a target area in a subsurface of the extremity. In one embodiment, the target area is the interior of a blood vessel. The method also includes angling the guide member to position the alignment feature on the guide member toward the target area. Angling the guide member may correspondingly move at least one imaging device coupled to the guide device so as to align the at least one imaging device to image the target area. In some embodiments, the at least one imaging device may include two or more imaging devices. The method can further include imaging the target area in the subsurface of the extremity using the at least one imaging device, displaying data representative of the at least one imaging device on a display in proximity to the extremity, and guiding a percutaneous access device along the guide member and into the target area. In some embodiments, the imaging is performed by an ultrasonic imaging system. In one embodiment, the displayed data is a three-dimensional representation of the target area displayed in real-time. In another embodiment, the displayed data is a two-dimensional representation of the target area displayed in real-time.

Another embodiment of the disclosure is an apparatus for positioning a vascular access device, comprising a guide device, at least two imaging devices, and an attachment device. In some embodiments, the guide device includes a leading edge, a base plate located in a plane, and a guide member pivotally coupled to the base plate at the leading edge. The attachment device can be attached to the base plate. The guide member can include an alignment feature. In some embodiments, the at least two imaging devices are positioned within the plane of the base plate and are configured to rotate axially as the guide member is pivotally angled. In one embodiment, the apparatus also includes a real-time imaging system coupled to the at least two imaging devices, wherein the imaging system is configured to receive data from the two imaging devices and display a representative image of the target area.

### BRIEF DESCRIPTION OF THE DRAWINGS

The foregoing and other features of the present disclosure will become more fully apparent from the following description taken in conjunction with the accompanying drawings. These drawings depict several examples of embodiments in accordance with the disclosure and are not to be considered limiting in their scope. The disclosure will be described with additional specificity and detail through use of the accompanying drawings.

FIG. 1 is a perspective view illustrating an embodiment of an apparatus for positioning a percutaneous access device configured to guide a needle for vascular access.

FIG. 2 is an enlarged perspective view of a portion of the embodiment shown in FIG. 1, further illustrating one example of an arrangement of imaging devices.

FIG. 3 is a side view of the embodiment shown in FIG. 1.

FIG. 4 is a front view of the embodiment shown in FIG. 1.

FIG. 5 is a top view of the embodiment shown in FIG. 1.

FIG. 6 is a flowchart illustrating one embodiment of a process for guiding insertion of a percutaneous access device.

### DETAILED DESCRIPTION OF CERTAIN EMBODIMENTS

The following detailed description is directed to certain implementations for the purposes of describing various innovative aspects. However, the teachings herein can be applied in a multitude of different ways. In the following detailed description, reference is made to the accompanying drawings, which form a part of the disclosure hereof. In the drawings, similar symbols typically identify similar components, unless context dictates otherwise. The illustrative embodiments described in the detailed description and drawings are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and comprise part of this disclosure.

The embodiments disclosed herein relate to medical procedures, generally, and more particularly to systems and methods for positioning a percutaneous access device. Such a system may guide a needle or other suitable instrument for insertion into a patient in a procedure related to, for example, percutaneous vascular access, needle aspiration, core biopsy, or nerve blockage. For clarity of this disclosure, any such needle or suitable instrument is referred to herein as a percutaneous access device. Some embodiments may comprise a positioning apparatus which is configured to guide a percutaneous access device along a portion of the positioning apparatus. The apparatus can be configured for coupling with one or more imaging devices. The imaging device(s) may be configured to produce real-time image data of a target area below the surface of the skin. In such embodiments, the image data can be communicated from imaging device(s) located within the positioning apparatus to an imaging system for display during percutaneous access device insertion procedures. The imaging device(s) may be movable (for example, rotatable) such that image data for multiple image planes can be collected and displayed. In some embodiments, movement of the imaging device(s) corresponds to movement of a guide member portion of the positioning apparatus, where the guide member is moved to align the percutaneous access device with a target. Such a target may include a blood vessel, lymph vessel, tumor, nerve, or other sub-surface anatomical feature. The coupled movement of the imaging device(s) with the guide member may move or rotate the imaging device(s) into a position to provide image data for an image plane that intersects with the percutaneous target.

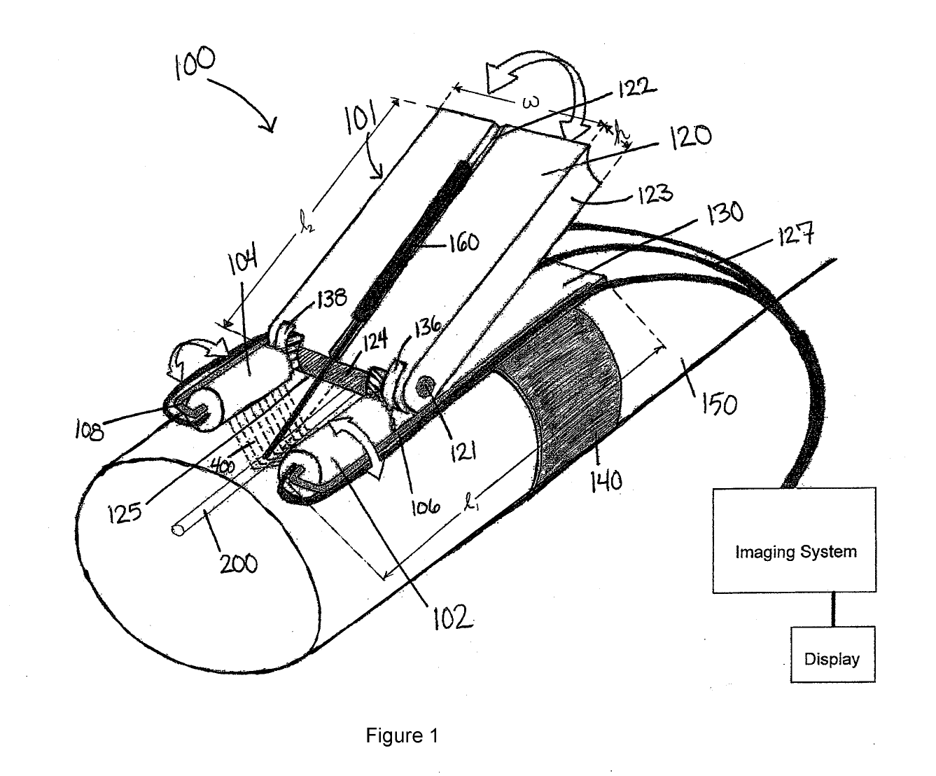

FIG. 1 illustrates a perspective view of one embodiment of an apparatus for positioning a percutaneous access device 100. The positioning apparatus 100 comprises a guide device 101 comprising a base plate 130 and guide member 120, and one or more imaging devices 102, 104.

In the embodiment of FIG. 1, the base plate 130 is configured to contact an extremity 150 (e.g., an arm or leg). In some embodiments, the base plate 130 can be configured to rest on and in proximity to an extremity without actually making contact. In other embodiments, the base plate 130 may rest on, in contact with or in proximity to, a torso, abdomen, or other anatomical feature. In FIG. 1, the base plate 130 is depicted as generally flat. However, in some embodiments the base plate 130 is not flat but instead at least slightly curved to generally fit, for example, the contour of the extremity 150. The base plate 130 can serve as a stabilizing structure for the apparatus 100. The base plate 130 can be secured to the extremity 150 with an attachment device, for example, a strap 140 attached to opposing sides of the base plate 130. In one embodiment, the base plate 130 includes one or more openings configured to receive attachment devices such as straps and belts. The strap 140 can include a Velcro attachment system, a clasp, snaps, or other fastening elements to secure the positioning apparatus 100 to the extremity 150. Other types of attachment devices or methods of securing the apparatus 100 to an extremity 150 can also be used. For example, a suitable material (e.g., double sided medical tape) can be disposed on the underside of the base plate 130 to increase the friction between the positioning apparatus 100 and the extremity 150 so as to prevent slippage. In some embodiments, the underside of the base plate 130 includes a roughened surface that increases friction but does not injure the extremity.

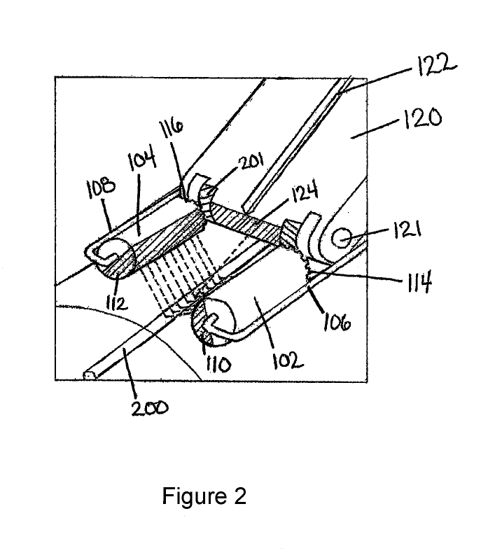

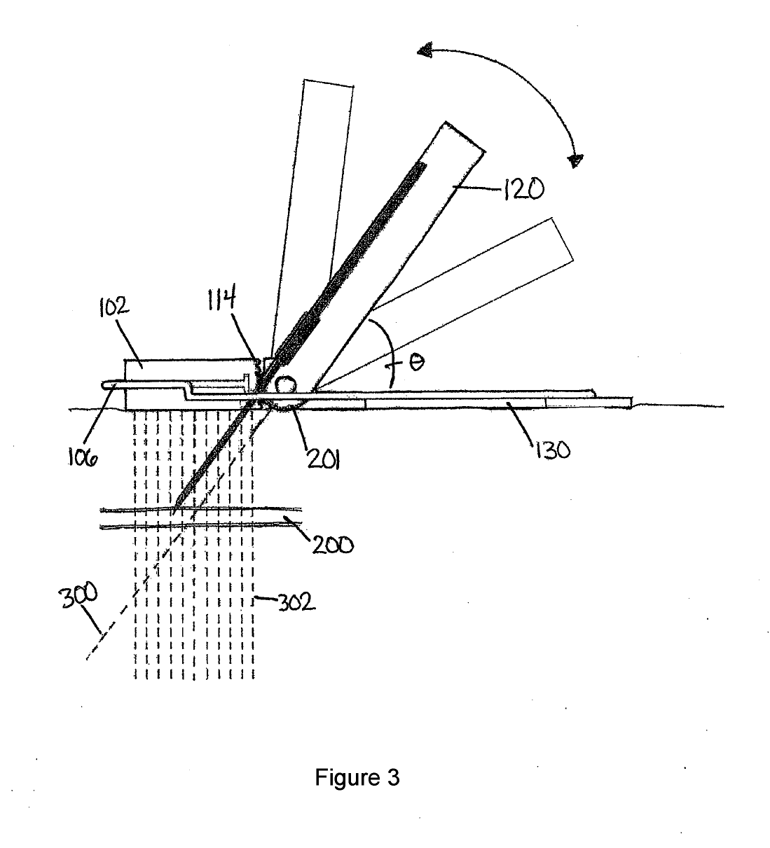

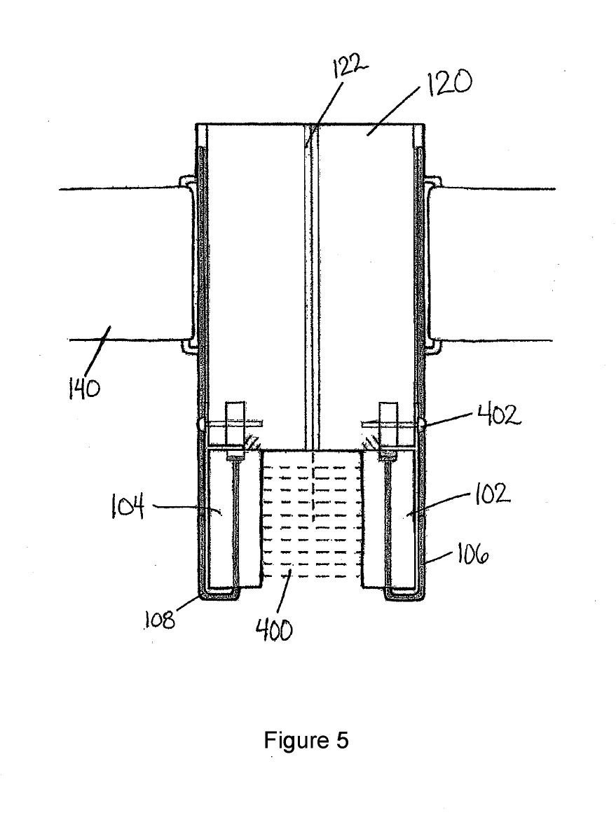

Referring to FIG. 2, which provides an enlarged perspective view of a portion of the embodiment shown in FIG. 1, the base plate 130 can be coupled to the guide member 120 at one end of the base plate 130 such that the guide member 120 moves relative to the base plate 130 in a hinge-like manner. The guide member 120 is configured to move about an axis of rotation located at or near a leading edge 125 of the guide device 101 (FIG. 1). The guide member 120 can be configured to move through a range of positions towards and away from the base plate 130. The range of positions is illustrated by angle θ in FIG. 3, where FIG. 3 depicts a side view of the embodiment shown in FIG. 1. In various embodiments, a percutaneous access device 160 may rest against the guide member 120 when the apparatus is in use, and the angle θ of the guide member 120 relative to the base plate 130 controls the percutaneous access device's angle of entry into a sub-surface target. In some embodiments, such as the one shown in FIG. 1, the base plate 130 includes receiving structures 136, 138 to couple the base plate 130 to a guide member 120. The receiving structures 136, 138 can be offset from the edges of the base plate 130 and can include holes for receiving pins 121 or other coupling structures. In some embodiments, one pin 121 travels through apertures in both the base plate 130 and guide member 120, thus coupling the base plate 130 and the guide member 120 so that they are operatively movable relative to each other. A locking mechanism can also be included such that the guide member 120 and base plate 130 can be temporarily fixed at a desired angle. Such a locking mechanism may include, without limitation, a thumb nut 402, as illustrated in FIG. 5. FIG. 5 provides a top view of the embodiment shown in FIG. 1.

Referring again to FIG. 1, in some embodiments the guide member 120 can be generally rectangular in shape with concave sides 123 that make the guide member 120 easy to grip and move. The top surface of the guide member 120 includes an alignment feature 122 for guiding a percutaneous access device 160 in a certain direction. In the illustrated implementation, the alignment feature 122 is disposed into a surface of the guide member 120. Also in this example, the alignment feature 122 is disposed in parallel to a longitudinal axis of the guide member 120. A portion, or all, of the alignment feature 122 can be exposed to allow the percutaneous access device 160 to lie in the alignment feature 122. In other implementations, the alignment feature can be one or more guide hooks, a notch, a tube, or a trough between two ridge structures. In the illustrated embodiment, the position of the alignment feature 122 is along a central portion of the guide member 120, however, in other embodiments, the alignment feature 122 can be positioned in another portion of the guide member 120, for example, in the surface but offset from the center of the guide member 120. The guide member 120 may also have an alignment feature on the underside or an axial bore hole for the placement of cables or wires 127, allowing the positioning apparatus 100 to be stored with the guide member 120 flat against the base plate 130 even when the cables or wires 127 are present. The positioning apparatus 100 is generally sized to be hand-held and may be further contoured to be easily grasped by hands. For example, in some embodiments, the positioning apparatus 100 is between about 3 cm and about 30 cm in length. In one embodiment, the guide member 120 is approximately 9 cm long, 4.5 cm wide, and 1 cm high/thick. In some embodiments, the base plate 130 is slightly larger than the guide member 120 and has a recessed center such that the guide member 120 is configured to collapse into and be stored within the base plate 130. In one embodiment, the base plate 130 is approximately 3 mm larger than the guide member 120 in each dimension (i.e., length, width, and height/thickness). One of skill in the art will appreciate that this disclosure encompasses additional embodiments wherein the guide member 120 and/or the base plate 130 comprise alternative sizes and shapes.

In order to aid a user in the insertion of a percutaneous access device, the positioning apparatus can also include at least one imaging device 102, 104, 124 configured to provide image data of percutaneous target areas and/or of the percutaneous access device. In certain embodiments, some or all of the imaging device(s) each include a housing configured to receive an imaging probe, such as the imaging devices illustrated in 102 and 104. In some embodiments, some or all imaging device(s) can include one or more imaging probes, as shown by imaging device 124. The imaging probe(s) can comprise ultrasonic probes for use with an ultrasound imaging system. In some embodiments, the ultrasonic probes are also capable of color flow Doppler interrogation, useful in targeting an artery, vein, or vascular structure. In other embodiments, the imaging probes include radio frequency electrodes for use with a magnetic resonance imaging system or other imaging probes known to those of skill in the art.

In one embodiment, the positioning apparatus does not include any imaging probes and is used as a mechanical guide alone, or it may be used with a separate non-coupled imaging probe to identify a target (e.g., a blood vessel) 200 and guide the percutaneous access device 160 to the target 200. In some embodiments, centering the positioning apparatus 100 over a target 200 can be achieved using one or more central imaging devices 124 located along and parallel to a leading edge 125 of the guide device 101. Such central imaging device(s) can be external to the guide member 120 and located within the same plane as the base plate 130 or can be on or housed within the guide member 120. In other embodiments, the positioning apparatus 100 includes one or more extended imaging devices 102, 104 extending from the base plate 130 or the guide member 120 at, for example, the leading edge 125. In various embodiments, the extended imaging devices 102, 104 are positioned to extend in a direction that is about perpendicular (i.e., about 90°) relative to the general direction defined by the leading edge 125. In one embodiment, the extended imaging devices 102, 104 can be positioned with an end spaced approximately 1 cm from the leading edge 125 and extending forward approximately 4-5 cm. Some embodiments, such as the embodiment of FIG. 1, include a combination of one or more central imaging devices 124 and one or more extended imaging devices 102, 104.

Referring to the embodiments illustrated in FIGS. 1-5, the two extended imaging devices 102, 104 are aligned along parallel axes offset from each other, abutting the guide member 120 within the same plane as the base plate 130. A central imaging device 124 is located parallel to the leading edge 125 of the guide member 120. The configuration of imaging devices in the illustrated embodiments is generally U-shaped. This configuration creates an open space on one end of the positioning apparatus, which may allow the apparatus 100 to be easily removed from the extremity 150 after a percutaneous access device 160 has been successfully inserted into the target 200. Additionally, this configuration forms a surface target area 400 between the extended imaging devices 102, 104. The surface target area 400 is sized to allow for the passage of small or large bore needles suitable for percutaneous vascular access, aspiration or biopsy of pathological structures, nerve blockages, or similar procedures.

The extended imaging devices 102, 104 can be stabilized by axial supports or struts 106, 108 which secure the extended imaging devices 102, 104 to the base plate 130 and allow for the extended imaging devices 102, 104 to rotate towards and away from the surface target area 400. The struts 106, 108 can be fixedly attached to the base plate 130 or comprise solid protrusions of the base plate 130. In some embodiments, the imaging devices 102, 104 are generally cylindrical in shape to facilitate rotation against an extremity 150. The central imaging device(s) 124 operatively move as the non-coupled portion of the guide member 120 moves through angle θ towards or away from the base plate 130. Additionally, the positioning apparatus 100 may be moved laterally or longitudinally on the extremity 150 to move the central imaging device(s) 124 in those respective directions.

Referring to FIG. 2, in some embodiments, the guide member 120 includes surfaces with toothed cogs 201 for operatively coupling with toothed cogs 114, 116 on the extended imaging devices 102, 104. The tooth cogs can include enmeshed tooth cogs. The toothed cogs 201 can be positioned on the guide member 120 near where the guide member 120 is attached to the base plate 130. The toothed cogs 114, 116 can be located on the ends of the extended imaging devices 102, 104 near the leading edge 125. In such embodiments, the interaction of the toothed cogs on the extended imaging devices 114, 116 with the toothed cogs on the guide member 201 allows for independent but coordinated rotation of the extended imaging devices 102, 104 in response to the angular movement of the guide member 120. While one example of operatively coupling the extended imaging devices 102, 104 is illustrated herein, other mechanical coupling structures, or electromechanical coupling structures can also be used to provide coordinated movement of the extended imaging devices 102, 104 with the guide member 120. For example, in another implementation, the extended imaging devices 102, 104 and/or the guide member 120 comprise one or more servo motors that are actuated to correspondingly rotate the extended imaging devices 102, 104 when the uncoupled portion of the guide member 120 is moved towards or away from the base plate 130.

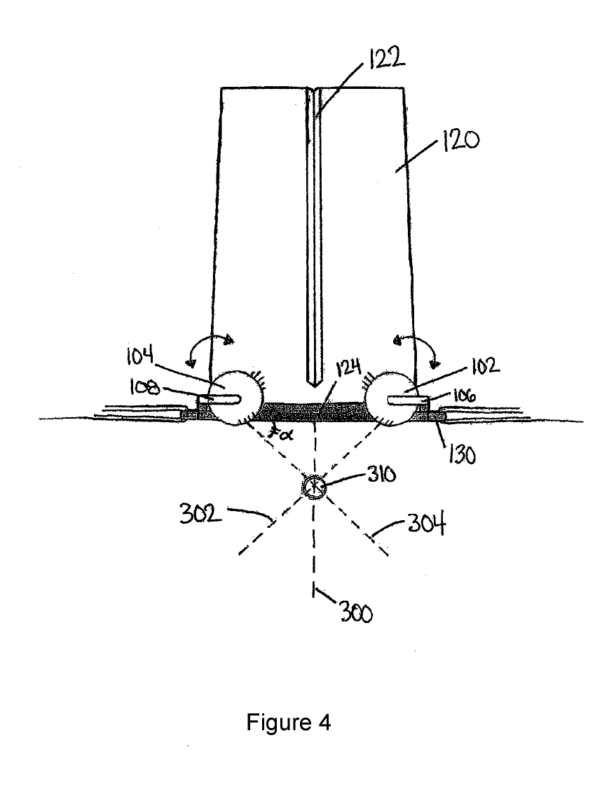

FIG. 4 illustrates a front view of the positioning apparatus embodiment shown in FIG. 1. FIG. 4 also schematically illustrates the central paths of an imaging beam (e.g., ultrasonic waves) emanating from the imaging probes 110, 112, 124, as arranged in this embodiment. Wave paths 300, 302, 304 represent a central direction of an imaging beam that respective imaging probes 124, 112, 110 may provide to image a target 200 at a location 310. The central wave path 300 emanates from the central imaging probe 124. The respective wave paths intersect at a location 310. In some examples, the imaging beam is an ultrasonic wave. Other suitable imaging systems can also be utilized in addition to, or instead of, ultrasonic imaging.

The angle α is defined relative to the base plate 130 and indicates the direction at which the wave paths 302, 304 (emanating from imaging probes 110, 112 located on extended imaging devices 102, 104) is changed as the guide member 120 moves relative to the base plate 130. For example, as the guide member 120 is moved away from the base plate 130 to provide a steeper angle of entry into the extremity 150, the angle α increases to provide deeper imaging. In this manner, the apparatus and accompanying imaging system can provide imaging of a sub-surface target along the path of entry of the percutaneous access device. Conversely, as the guide member 120 is moved towards the base plate 130 such that the guided angle of entry of the percutaneous access device is at a shallower angle, the angle α decreases to image closer to the surface and again target along the path of entry of the percutaneous access device.

The base plate 130, guide member 120, extended imaging devices 102, 104, toothed cogs 114, 116, 201 and struts 106, 108 can be made with molded plastic, suitable metals, composites, or other similar materials. Different components can be made of different materials. In one embodiment, the positioning apparatus can be entirely disposable. The positioning apparatus can be constructed of polyolefin plastic or other disposable, medical grade plastic or composite. In such an embodiment, the imaging device(s) can comprise a housing which fully engulfs and protects the imaging probe(s) they are configured to receive. Such imaging probe(s) may be slid or clipped into place. In some embodiments, the housing can be configured to open in a clamshell-like manner facilitating protective placement of the imaging probe(s). A variety of styles can be used for the configuration of the housing to match the various sizes and wiring needs of available imaging probes. In other embodiments, the housing includes a strut or axial support on which an imaging probe may be clipped. In such embodiments, the imaging probe can be disposable or can be wrapped in a disposable film or covering. In still other embodiments, all or some components of the positioning apparatus are reusable. In such embodiments, the reusable components are made of a durable material capable of withstanding sterilization methods such as autoclaving, liquid immersion, or steam exposure. The reusable components can be made of a durable metal, polyphenylsulfone, or other suitable material known to those of skill in the art.

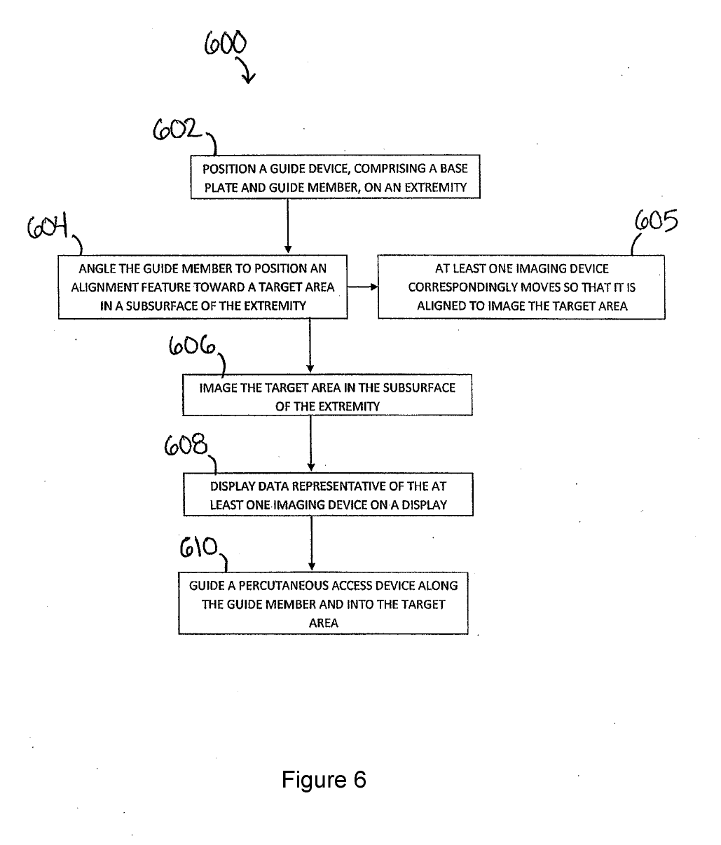

The apparatus for positioning a percutaneous access device may be used according to certain embodiments, including the embodiment described in the flowchart depicted in FIG. 6. An example of such a positioning apparatus 100 is illustrated in FIGS. 1-5. The devices and components described in reference to FIG. 6 are also illustrated in one or more of FIG. 1-5. The positioning apparatus can be powered on to activate one or more of imaging devices included in the apparatus. At block 602, the positioning apparatus 100 is placed on an extremity 150 in order to accurately locate a percutaneous target 200 such as a blood vessel. The extremity 150 may be covered with ultrasonic gel. To position the apparatus 100, it can be moved laterally and longitudinally about the surface of the extremity 150.

At block 604, an uncoupled portion of the guide member 120 can be moved towards and away from the base plate 130 by a user. At block 605, this angular movement of the guide member 120 causes the extended imaging devices 102, 104 and/or the central imaging device 124 to move correspondingly with the movement of the guide member 124. For example, in some embodiments, as the incline of the guide member 120 increases, such that the central imaging device(s) 124 and percutaneous access device 160 are directed towards a deeper target area in the extremity 150, the extended imaging device(s) 102, 104 rotate. Rotation of the extended imaging device(s) 102, 104 causes the imaging wave paths 302, 304 to intersect at a deeper percutaneous location. Conversely, in some embodiments, the guide member 120 can be moved to a shallower angle relative to the base plate 130 causing the extended imaging device(s) 102, 104 to rotate and point to a superficial target region similar to that which the central imaging device(s) 124 and percutaneous access device 160 also aim. This coordinated movement allows for all of the imaging devices positioned on the apparatus 100 to focus towards the same target area. At block 606, at least one imaging device images the target area in the subsurface of the extremity, and at block 608, an imaging system displays data representative of the at least one device on a display.

The imaging device(s) can be wired to a suitable imaging processor, such as an ultrasound system processor. In some embodiments, at least one ultrasonic probe sends ultrasonic beams of energy along an image plane into a percutaneous target area, receiving echo signals in return. In one embodiment, the ultrasonic probes use a high frequency (8-10 mHz) signal. Data from the echo signals is sent to an ultrasound system processor for processing. Image data received from one image probe can be processed and displayed graphically or as a two-dimensional image in real time. In some embodiments where two imaging devices 102, 104 share the same target focal area, image data is processed from imaging probes within each imaging device, and biplane imaging may be achieved. With the use of software, images from two imaging probes 110, 112 may be rapidly and alternately projected onto a single screen. The image of the target area from two vantage points can be centered relative to the alignment feature 122 of the guide member 120. Further, combining images with a centerline along the central axis of the individual images generated from each imaging probe can be used to confirm equivalent targeting of the oppositely directed images. Those of skill in the art will appreciate that with the proper configuration of imaging probes and appropriate software, three-dimensional images of percutaneous anatomical structures may be generated. Additionally, in some embodiments, the processor and display unit can be configured to display color Doppler images that may depict the relative speeds of fluids traveling in target vessels and assist in identifying the target.

A percutaneous access device 160 can be placed within the alignment feature 122. A user can maneuver the apparatus 100 with one hand while maneuvering the percutaneous access device 160 with the other hand while also observing the image displayed. Once the target 200 is identified, the apparatus 100 can be secured to the patient, the locking mechanism may fix the guiding member 120 at a desired angle θ, and, as at block 610, the percutaneous access device 160 can be guided along the guide member and inserted into the target area of the extremity 150. The depth of the percutaneous access device 160 is then monitored and the percutaneous access device may be stopped when the target 200 is reached. In order to facilitate visualization of the depth of the percutaneous access device, some embodiments may use specialized needles having one or more electromagnetic sensors embedded into the needle tip. Such a needle can provide additional signaling data to the imaging processor, and the displayed image can be updated to show a line projection through the image simulating the course of the needle through the soft tissue. In some such embodiments, image data from the additional vantage point may allow a user to determine the accurate distance from the skin surface to the target 200.

After the percutaneous access device has been properly placed into the target 200, the apparatus 100 can be slid away and removed from the patient without disturbing the location of the percutaneous access device.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or” in reference to a list of two or more items covers all of the following interpretations of the word: any of the items in the list, all of the items in the list, and any combination of the items in the list. The word “exemplary” is used exclusively herein to mean “serving as an example, instance, or illustration.” Any implementation described herein as “exemplary” is not necessarily to be construed as preferred or advantageous over other implementations.

The disclosure is not intended to be limited to the implementations shown herein. Various modifications to the implementations described in this disclosure may be readily apparent to those skilled in the art, and the generic principles defined herein may be applied to other implementations without departing from the spirit or scope of this disclosure. The teachings of the invention provided herein can be applied to other methods and systems, and are not limited to the methods and systems described above, and elements and acts of the various embodiments described above can be combined to provide further embodiments. Accordingly, the novel methods and systems described herein may be embodied in a variety of other forms; furthermore, various omissions, substitutions and changes in the form of the methods and systems described herein may be made without departing from the spirit of the disclosure. The accompanying claims and their equivalents are intended to cover such forms or modifications as would fall within the scope and spirit of the disclosure.

What is claimed is:

1. An apparatus for positioning a percutaneous access device, comprising:
a guide device comprising
a base plate;
a guide member having a first surface and a second surface opposite the first surface, the guide member pivotally coupled to the base plate such that the second surface is proximal to the base plate, the guide member comprising an alignment feature disposed on the first surface and configured to receive and guide a percutaneous access device along a first direction on the guide member towards a target point; and
an attachment device coupled to the base plate to removably secure the guide device to an extremity.

2. The apparatus of claim 1, further comprising at least one imaging device coupled to the guide device, the at least one imaging device positioned to image an area around the target point.

3. The apparatus of claim 2, wherein the at least one imaging device is rotationally coupled to the guide device.

4. The apparatus of claim 2, wherein the at least one imaging device comprises an ultrasonic probe positioned at a leading edge of the guide device.

5. The apparatus of claim 2, wherein the at least one imaging device comprises two imaging devices disposed apart from each other, wherein the two imaging devices are both configured to image an area around the target point each from a different position.

6. The apparatus of claim 5, wherein the two imaging devices are coupled to the guide device, and wherein the two imaging devices are configured to move as the guide member is pivotally moved relative to the base plate, the movement of the imaging devices changing the imaging area of each device.

7. The apparatus of claim 5, wherein each of the two imaging devices comprises a housing for holding an imaging probe.

8. The apparatus of claim 5, wherein each of the two imaging devices comprises an ultrasonic probe positioned at a leading edge of the guide device.

9. The apparatus of claim 5, wherein the coupling of the two imaging devices to the guide device comprises a plurality of toothed cogs configured to engage a plurality of toothed cogs on the guide device.

10. The apparatus of claim 5, wherein the coupling of the two imaging devices to the guide device comprises a plurality of toothed cogs configured to engage a plurality of toothed cogs on the guide device.

11. The apparatus of claim 1, wherein the attachment device comprises a strap.

12. The apparatus of claim 1, wherein the alignment feature is a groove.

13. The apparatus of claim 1, wherein the alignment feature is disposed in a direction parallel to a longitudinal axis of the guide member.

14. The apparatus of claim 1, wherein the alignment feature is one of at least one guide hook, a notch, a tube, a trough between two ridges, and a groove.

15. A method of guiding insertion of a percutaneous access device comprising:
positioning a guide device comprising a base plate coupled to a guide member on an extremity, wherein the base plate is positioned against the extremity;
angling the guide member to position an alignment feature on the guide member toward a target area in a subsurface of the extremity, wherein angling the guide member correspondingly moves at least one imaging device coupled to the guide device such that the at least one imaging device is aligned to image the target area;
imaging the target area in the subsurface of the extremity using the at least one imaging device;
displaying data representative of the at least one imaging device on a display in proximity to the extremity; and
guiding a percutaneous access device along the guide member and into the target area.

16. The method of claim 15, wherein the target area is the interior of a blood vessel.

17. The method of claim 15, wherein the imaging is performed by an ultrasonic imaging system.

18. The method of claim 15, wherein the at least one imaging device comprises two imaging devices, and the displayed data is a three-dimensional representation of the target area displayed in real-time.

19. An apparatus for positioning a vascular access device, comprising:
a guide device comprising:
a leading edge,
a base plate located in a plane, and
a guide member pivotally coupled to the base plate at the leading edge,
the guide member comprising an alignment feature;
at least two imaging devices each positioned within the plane, the at least two imaging devices configured to rotate axially as the guide member is pivotally angled; and
an attachment device attached to the base plate.

20. The apparatus of claim 19, further comprising a real-time imaging system coupled to the at least two imaging devices, the imaging system configured to receive data from the two imaging devices and display a representative three-dimensional image of the target area.
